(19) United States
(12) Patent Application Publication
(10) Pub. No.: US 2009/0292205 A1
(43) Pub. Date: Nov. 26, 2009

(54) ULTRASONIC DIAGNOSTIC APPARATUS

(76) Inventor: Takashi Osaka, Tokyo (JP)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 12/374,081

(22) PCT Filed: Jul. 18, 2007
(86) PCT No.: PCT/JP2007/064134

§ 371 Date: Jan. 16, 2009

(30) Foreign Application Priority Data
Jul. 18, 2006 (JP) 2006-195173

(51) Int. Cl. A61B 8/14

(52) U.S. Cl. 600/443

(57) ABSTRACT

In an ultrasonic diagnostic apparatus comprised of an ultrasonic probe; frame data acquiring means for time sequentially acquiring a plurality of RF signal frame data in a process of change in a pressure state of a subject tissue of an object to be examined while pressing the ultrasonic probe on the object tissue; and elasticity information calculating means for deriving a pair of data from RF frame signal data, calculating each place of distortion or elasticity modulus of the object tissue, and generating a plurality of elastic frame data; elastic image construction means for adding the plurality of elasticity frame data and generating an elastic image; and display means for displaying the elastic image, the ultrasonic diagnostic apparatus is further provided with evaluation means for evaluating the reliability of the plurality of the elasticity frame data subjected to adding in accordance with the degree of pressure.

### TECHNICAL FIELD

The present invention is related to an ultrasonic diagnostic apparatus comprising a function for generating elasticity information such as an elastic image indicating hardness or softness of tissues based on strain of tissues, etc. upon applying pressure to the tissues.

### BACKGROUND ART

In an elasticity imaging by an ultrasonic diagnostic apparatus, compression (pressure) is applied to an object to be examined by an ultrasonic probe using a manual or mechanical method, and elasticity information such as strain or elasticity modulus indicating hardness or softness of tissues is obtained and displayed based on the displacement of each area between two RF frame data (ultrasonic tomographic image) measured at different times.

In elasticity imaging, it is considered clinically crucial to display the most appropriate elastic image at any given time, since compressed condition of the object changes in real time and imaging condition changes during the process. For example, in the case that displacement between two RF frame data is small when light pressure change is applied, since the strain amount to be acquired by the two RF frame data is small, valuable information could be buried in an error (noise, etc.) generated in the RF frame data. Contrarily, in the case that displacement between the two RF frame data turns out to be large with light pressure change due to low compression from the probe to the object, since the interval between timings of acquiring the two RF frame data become large and direction of pressure to be applied to the target region of the object changes, unreliable elastic images could be constructed.

In Patent Document 1 (column 13, line 3), the method is disclosed for calculating quality factor (Qk) based on the difference between adjacent elasticity frame data in chronological order (δk) or the average value in a predetermined matrix of the elasticity frame (μₖ), and constructing an image, in accordance with the size of the calculated value, by weighting and adding the calculated value on the elastic image of the frame generated previously to the relevant elastic image.

Patent Document 1: U.S. Pat. No. 6,558,324B1

In the technique disclosed in the above-mentioned Patent Document 1, the above-described desired calculation process is performed on elasticity frame data in order to evaluate reliability of tomographic images.

However, reliability of elastic images is often influenced by factors other than the ones that can be evaluated by determination of parameter Qk (degree of data value in elasticity frame data), for example, pressure to be added to an object from a probe or imaging condition, etc. Yet those factors such as the pressure to be added from the probe to the object or imaging condition are not considered in the above-mentioned Patent Document 1.

### DISCLOSURE OF THE INVENTION

### Problems to be Solved

The objective of the present invention is to provide an ultrasonic diagnostic apparatus capable of evaluating elasticity information to be displayed in consideration of factors such as the degree of pressure being added from the probe to the object, etc. and displaying the most adequate elastic image in accordance with the evaluation result thereof.

In accordance with the present invention, the ultrasonic diagnostic apparatus comprises:

an ultrasonic probe;

frame data acquiring means for applying pressure to target tissues of an object to be examined via the ultrasonic probe, and acquiring a plurality of RF signal frame data in chronological order in the process that compression condition of the target tissues changes;

elasticity information calculating means for deriving a pair of RF signal frame data from among the plurality of RF signal frame data, calculating strain or elasticity modulus of each position of the target tissues, and generating a plurality of elasticity frame data;

elastic image constructing means for constructing an elastic image by adding the plurality of elasticity frame data; and

display means for displaying the elastic image,

and is characterized in further comprising:

evaluation means for evaluating reliability of the plurality of elasticity frame data to be added based on the degree of compression.

The ultrasonic diagnostic apparatus of the present invention also comprises adjusting means for adjusting addition of the plurality of elasticity frame data in accordance with the evaluation result by the evaluation means.

The adjusting means adjusts addition by varying the weighting of elasticity frame data to be the subject of the addition.

### EFFECT OF THE INVENTION

In accordance with the present invention, it is possible to provide an ultrasonic diagnostic apparatus capable of evaluating displayed elastic images in consideration of factors such as the degree of compression applied from a probe to an object to be examined, and displaying the most adequate elastic image in accordance with the evaluation result thereof.

### BRIEF DESCRIPTION OF THE DIAGRAMS

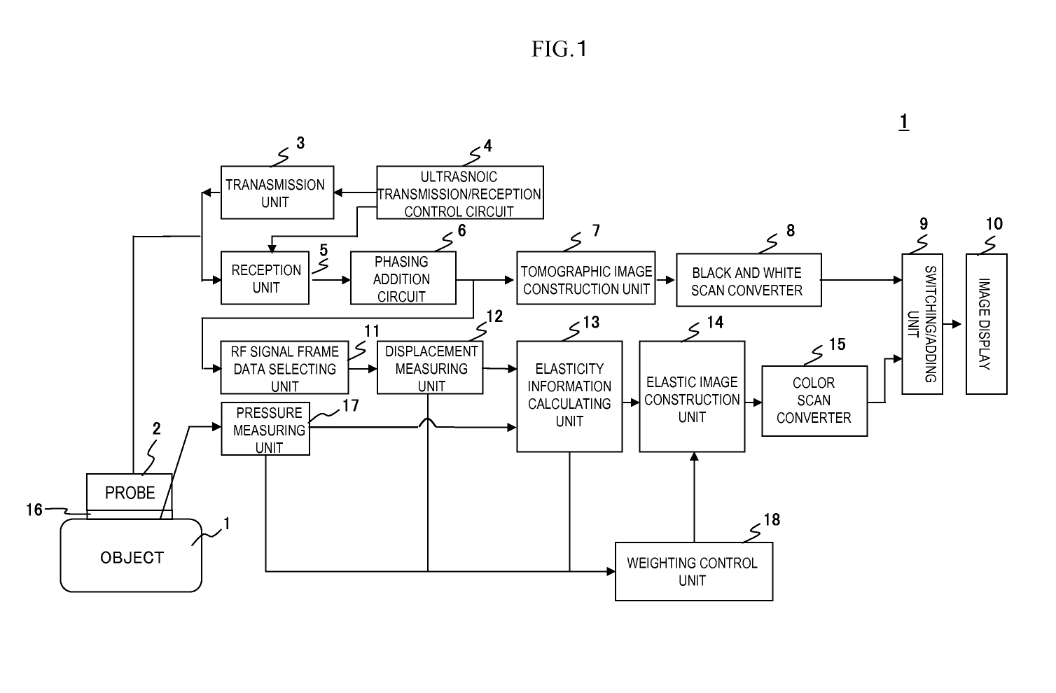

FIG. 1 is a block configuration diagram of a first embodiment of the ultrasonic diagnostic apparatus related to the present invention.

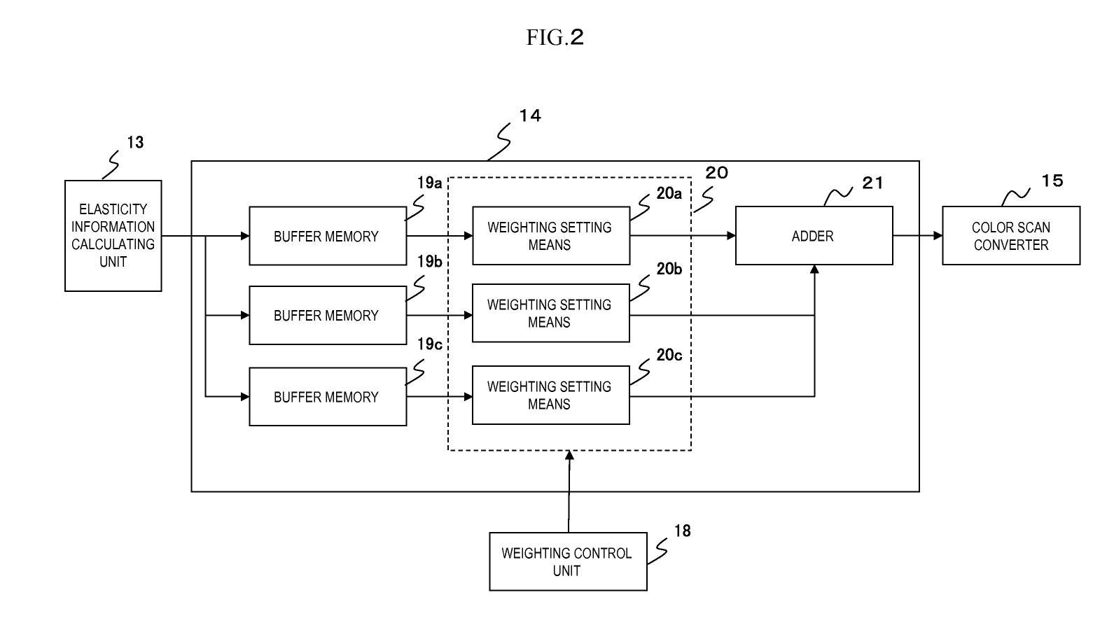

FIG. 2 illustrates details of the first embodiment related to the present invention.

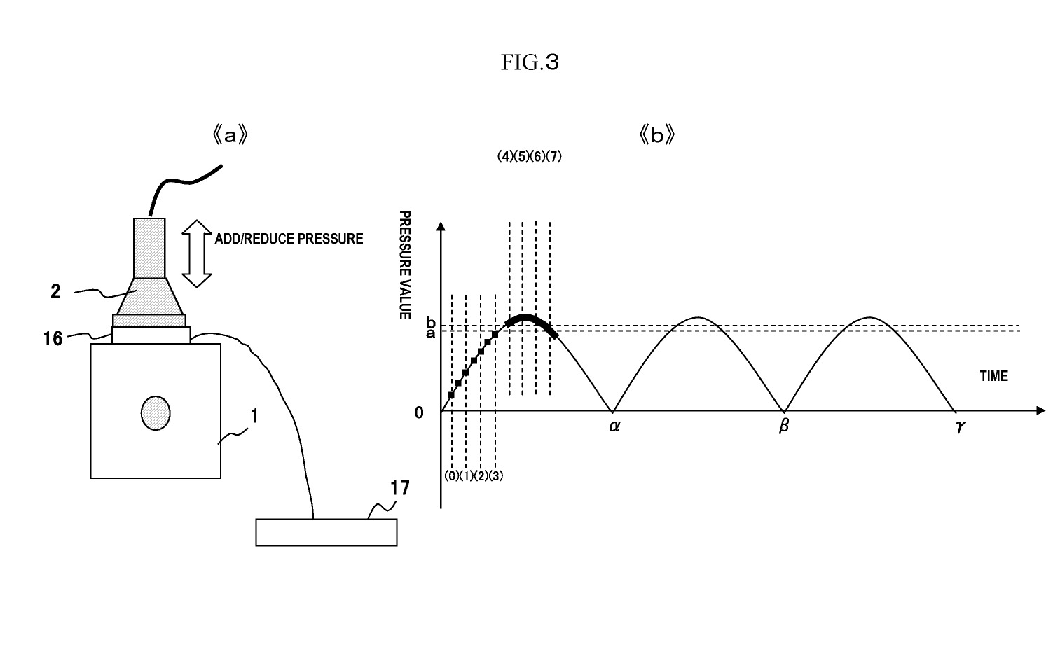

FIG. 3 is a concrete example of weighting in the first embodiment related to the present invention.

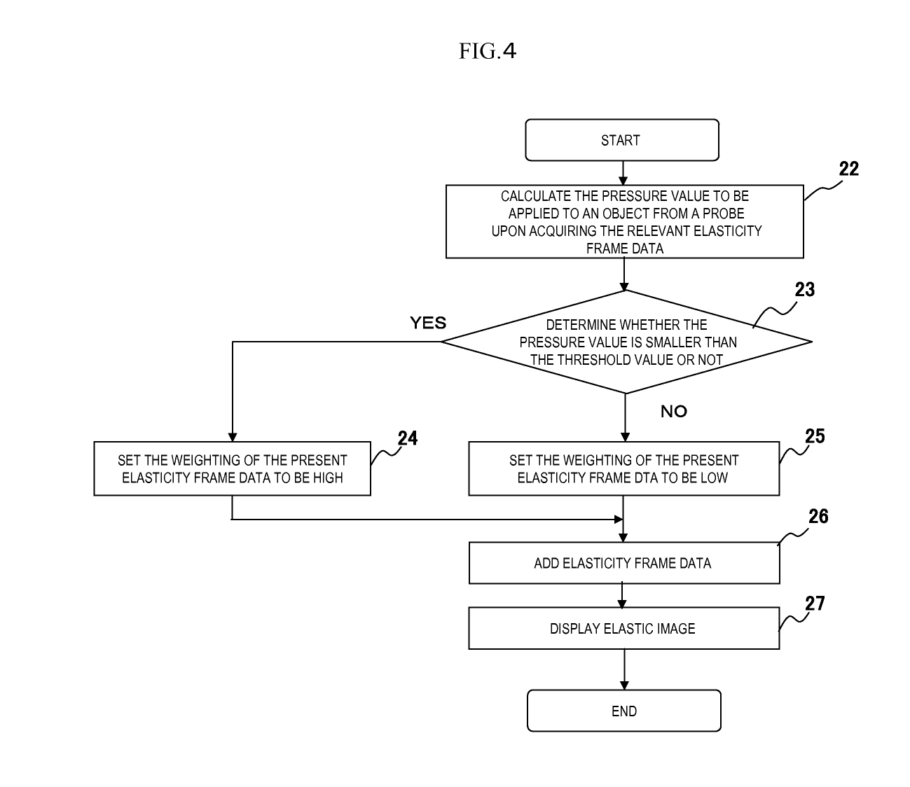

FIG. 4 illustrates operation of the first embodiment related to the present invention.

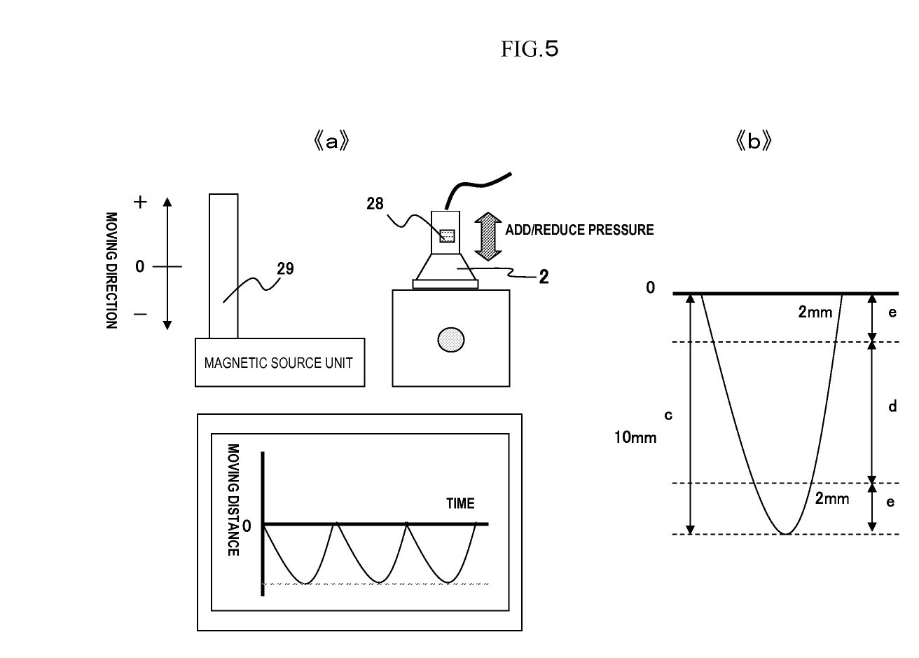

FIG. 5 illustrates a second embodiment of the ultrasonic diagnostic apparatus related to the present invention.

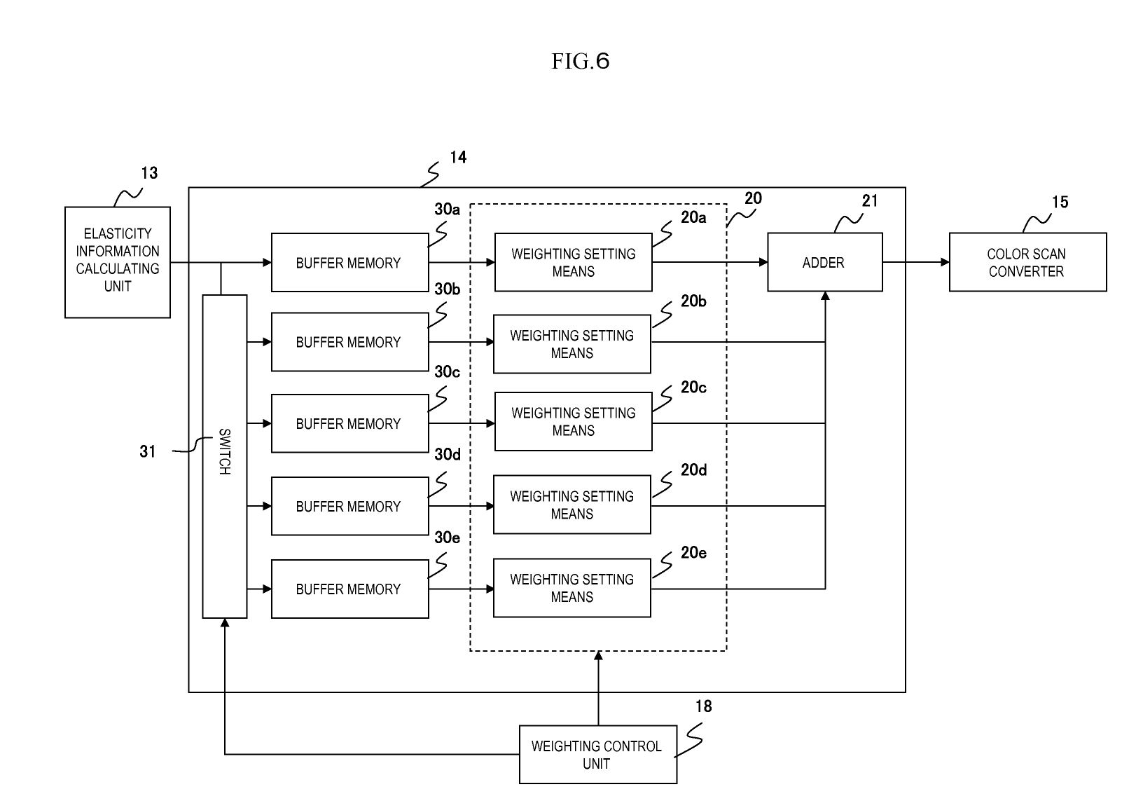

FIG. 6 illustrates a third embodiment of the ultrasonic diagnostic apparatus related to the present invention.

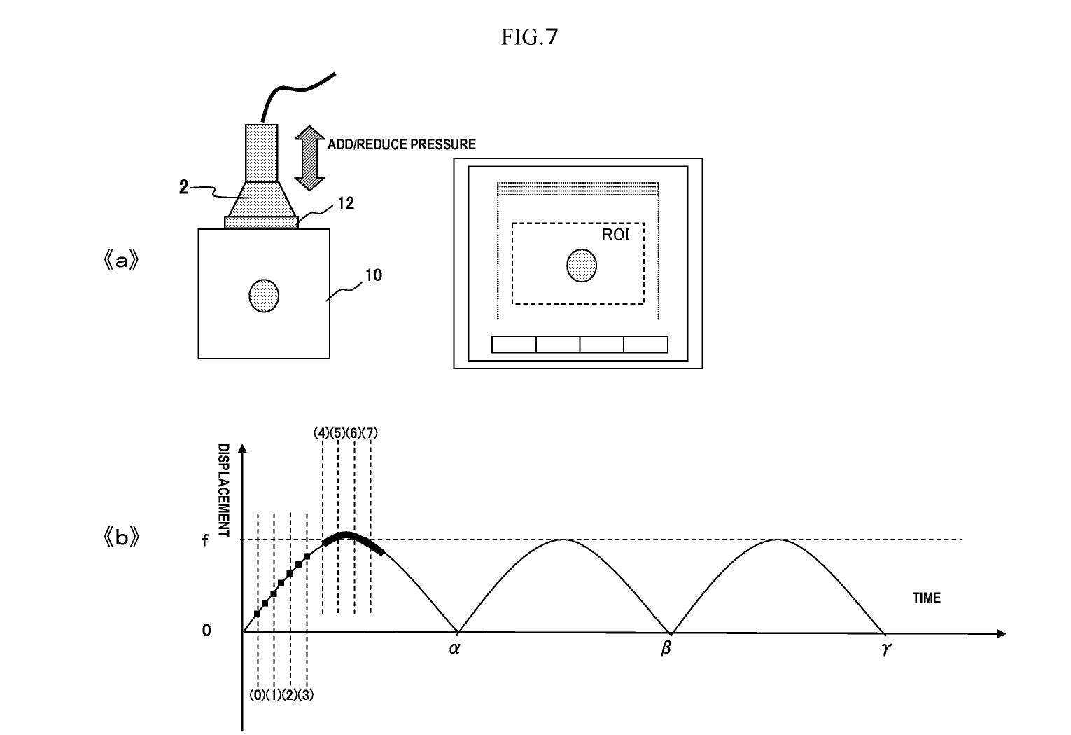

FIG. 7 illustrates a fourth embodiment of the ultrasonic diagnostic apparatus related to the present invention.

### DESCRIPTION OF THE SYMBOLS

13 . . . elasticity information calculating unit, 14 . . . elastic image construction unit, 15 . . . color scan converter, 18 . . . weighting control unit, 19a˜c . . . buffer memory, 20a˜c . . . weighting setting means, 21 . . . adder

### BEST MODE FOR CARRYING OUT THE INVENTION

Hereinafter, embodiments of the present invention will be described referring to the diagrams.

### Embodiment 1

FIG. 1 is a block configuration diagram of the first embodiment related to the ultrasonic diagnostic apparatus of the present invention.

As shown in FIG. 1, an ultrasonic probe 2 for applying to an outer surface of the object 1 is configured having an ultrasonic transmitting/receiving surface in which a plurality of transducers are arranged for transmitting/receiving ultrasonic waves to/from the object 1. The ultrasonic probe 2 is connected to a transmission unit 3, and the transmission unit 3 provides ultrasonic pulses to the probe 2 for driving the probe 2. An ultrasonic transmission/reception control circuit 4 is connected to the transmission unit 3 and a reception unit 5 to be described later, and the ultrasonic transmission/reception control circuit 4 controls transmission timing of ultrasonic pulses for driving a plurality of transducers in the probe 2 to form ultrasonic beams toward a focal point to be set in an object 1. The ultrasonic transmission/reception circuit 4 controls to electronically scan ultrasonic beams in the array direction of transducers of the probe 2.

On the other hand, the reception unit 5 is also connected to the probe 2, and the probe 2 receives the reflected echo signals generated from the object 1 and outputs the received signals to the reception unit 5. The reception unit 5 acquires the reflected echo signals in accordance with the transmission timing of the ultrasonic pulses controlled by the ultrasonic transmission/reception control circuit 4, and performs reception process such as signal amplification. A phasing addition circuit 6 is connected to the reception unit 5, and the phasing addition circuit 6 performs phasing addition, to amplify the signals, on the reflected echo signals that are performed with reception process by the reception unit 5. A tomographic image construction unit 7 is connected to the phasing addition circuit 6, and the tomographic image construction unit 7 performs signal processing such as gain compensation, log compression, detecting phase, edge enhancement and filtering on the RF signals of the reflected echo signals phased and added by the phasing addition circuit 6 to obtain tomographic image data. Also, a black and white scan converter 8 is connected to the tomographic image construction unit 7, and the black and white scan converter 8 converts the RF signals processed in the tomographic image construction unit 7 into digital signals and converts them into 2-dimensional tomographic image data corresponding to the scan plane of ultrasonic beams. Image construction means of tomographic images (B-mode) is configured by the above-mentioned tomographic image construction unit 7 and the black and white scan converter 8. The tomographic data outputted from the black and white scan converter 8 is provided to the image display unit 10 via the switching/adding unit 9 to be described later and the B-mode images are to be displayed thereto.

At the same time, an RF signal frame data selecting unit 11 is connected to the phasing addition unit 6, and the RF signal frame data selecting unit 11 obtains the RF signal group to correspond to the scan plane (tomographic plane) of ultrasonic beams for the portion of a plurality of frames as RF signal frame data, and stores the obtained signal group in a device such as memory.

More specifically, the RF signal frame data selecting unit 11 stores the plurality of RF signal frame data from the phasing addition circuit 6, and selects a pair of, that is two sets of RF signal frame data from the stored RF signal frame data group. For example, while it sequentially records RF signal frame data generated in chronological order from the phasing addition circuit 6 and selects the newest recorded RF signal frame data (N) as the first data, it also selects one set of RF signal frame data (X) from among the RF signal frame data group (N-1, N-2, N-3, . . . , N-N′) recorded temporally in the past. Here, N, N′ and X are index appended to RF signal frame data, and are whole numbers.

A displacement measuring unit 12 is connected to the RF signal frame data selecting unit 11, and the displacement measuring unit 12 sequentially loads plural pairs of frame data acquired at different times that are stored in the RF signal frame data selecting unit 11, obtains a displacement vector of a plurality of measuring points in the tomographic planes based on a loaded pair of frame data, and outputs them as displacement frame data to an elasticity information calculating unit 13 to be described later.

More specifically, the displacement measuring unit 12 performs one-dimensional or two-dimensional correlation process from a pair of selected data that is RF signal frame data (N) and RF signal frame data (X), and obtains displacement or movement vector in the biological tissue corresponding to the respective points of a tomographic image that is one-dimensional or two-dimensional displacement distribution related to the direction and size of the displacement. Here, the block matching method is to be used for detecting a movement vector. The block matching method divides an image into, for example, blocks formed by N×N pixels, focuses attention to the block in the region of interest, search for the most approximated block to the focused block from the previous frame, and calculates displacement by acquiring the difference by referring to the searched block.

The elasticity information calculating unit 13 is connected to the displacement measuring unit 12, and the elasticity information calculating unit 13 is configured having function for generating frame data on change of strain by obtaining change of strain in the tissue of each measuring point based on the displacement frame data or the function for calculating other elasticity information (such as elasticity modulus, viscosity coefficient, strain, stress, strain ratio and Poisson's ratio).

For example, data of strain change is calculated by performing spatial differentiation on moving distance such as displacement of the biological tissues. Also, data of elasticity modulus is calculated by, for example, dividing pressure change by strain change. For example, when the displacement calculated by the displacement measuring unit 12 is set as L(i,j) and the pressure measured by a pressure measuring unit 18 to be described later is set as P(i,j), strain change ΔS(i,j) can be calculated by performing spatial differentiation on L(i,j) using a formula of ΔS(i,j)=ΔL(i,j)/ΔX(i,j), and elasticity modulus can be calculated by a formula of Ym=ΔP(i,j)/ΔS(i,j). Since elasticity modulus of the biological tissues which is equivalent to the respective points in the tomographic image can be obtained from the above-described elasticity Ym, it is possible to acquire two-dimensional elasticity frame data in real time.

An elastic image construction unit 14 is connected to the elasticity information calculating unit 13, and the elastic image construction unit 14 is configured including a buffer memory and an image processing unit. It records the elasticity frame data outputted from the elasticity information calculating unit 13 in time chronological order to the buffer memory, and performs a variety of image processing such as smoothing process and contrast optimization process in the plane with a coordinate system, and smoothing process in time axis direction between the frames with respect to the recorded elasticity frame data.

A color scan converter 15 is connected to the elastic image construction unit 14, and the color scan converter 15 loads frame data of elasticity information outputted from the elastic image construction unit 14 and appends hue code for each pixel of the frame data in accordance with the color map of the set elasticity information so as to construct color images. More specifically, the color scan converter 15 converts image data into 3 primary colors of light that are red (R), green (G) and blue (B) based on the elasticity frame data so as to construct a display screen of an elasticity image. For example, it converts elastic image data having large strain into red color code, as well as converting the elastic image data having small strain into blue color code.

The color elastic images constructed by the color scan converter 15 are to be displayed on the image display unit 10 via the switching/calculating unit 9.

The switching/calculating unit 9, when the black and white tomographic image outputted from the black and white scan converter 8 and the color elastic image outputted from the color scan converter 15 are inputted, has a function for switching the images to display one of them, a function for making one of the images translucent to perform additive synthesis and displaying them on the image display 10 as a composite image, and a function for juxtaposing and displaying both images. More concretely, the switching/calculating unit 9 is configured comprising a frame memory, an image processing unit and an image selecting unit. Here, the frame memory is for storing black and white tomographic images from the black and white scan converter 8 and color elastic images from the color scan converter 15. The image processing unit is for synthesizing a black and white tomographic image recorded in the frame memory and a color elastic image at an arbitrary composite rate so as to construct a composite image. The image selecting unit is for selecting an image for displaying on the image display unit 10 from among the black and white tomographic images and the color tomographic images in the frame memory and the composite images constructed by the image processing unit.

Further, the ultrasonic diagnostic apparatus related to the first embodiment of the present invention is provided with a pressure sensor 16 between the object 1 and the probe 2. The technique related to the pressure sensor in the ultrasonic diagnostic apparatus is disclosed in a document such as Patent Document JP-A-2004-261198 in the paragraph of [0049]. The signals from the pressure sensor 16 are transmitted to a pressure measuring unit 17 connected to the pressure sensor 16, and the pressure measuring unit 17 calculates the pressure value added to the object 1 from the probe 2 based on the electronic signals from the pressure sensor 16 and transmitted to an elasticity information calculation unit 13.

The weighting control unit 18 is connected, as to be described later, to the devices such as displacement measuring unit 12, elasticity information calculating unit 13, pressure measuring unit 17, ultrasonic transmission/reception control circuit 4 though not shown in the diagram, phasing addition circuit 6 and elastic image construction unit 14, and is for performing weighting upon adding elasticity frame data or control of the number of addition with respect to the elastic image construction unit 14 in accordance with frame rate of the elastic images obtained in the respective components or compression information added from the ultrasonic probe 2 to the object.

Next, the details of the first embodiment in the present invention will be described using FIG. 2. FIG. 2 shows the configuration of the elastic image construction unit 14 illustrated in FIG. 1.

The elastic image construction unit 14 is configured by a plurality of buffer memories 19a˜c for recording the elasticity frame data acquired from the elasticity information calculating unit 13, weighting setting means 20a˜c to perform weighting corresponding to the respective plurality of buffer memories, and the adder 21 for generating one set of elastic image data by adding the plurality of elastic frame data in accordance with the weight thereof respectively.

The elasticity frame data acquired in the elasticity information calculating unit 13 is sequentially recorded for the portion of 3 sets of frames in the buffer memory 19a, buffer memory 19b and buffer memory 19c. For example, when the newest elasticity frame data recorded in the buffer memory 19a is set as elasticity frame data “N”, elasticity frame data “N-1” is recorded in the buffer memory 19b and elasticity frame data of the frame “N-2” is sequentially recorded in the buffer memory 19c respectively in chronological order.

The weighting setting means 20a is connected to the buffer memory 19a, and performs weighting on the elasticity frame data “N” recorded in the buffer memory 19a by the weighting control unit 18 to be described later. The weighting setting means 20b is connected to the buffer memory 19b, and performs weighting on the elasticity frame data “N-1” recorded in the buffer memory 19b by the weighting control unit 18. The weighting setting means 20c is connected to the buffer memory 19c, and performs weighting on the elasticity frame data “N-2” recorded in the buffer memory 19c by the weighting control unit 18.

The weighting control unit 18 is connected to the weighting setting means 20a˜c. The weighting control unit 18 is connected to the devices such as the displacement measuring unit 12, elasticity information calculating unit 13, pressure measuring unit 17, and ultrasonic transmission/reception control circuit 4 and phasing addition circuit 6 though not shown in the diagram.

The weighting control unit 18 controls the weight of the weighting setting means 20a˜c in accordance with factors such as compression information added to the skin of the object 1 from the ultrasonic probe 2 or the frame rate of the elastic image obtained in the respective components. The weighting setting means 20a˜c performs weighting on the respective sets of elasticity frame data and outputs the weighted data to the adder 21. The adder 21 adds three sets of elasticity frame data outputted respectively from the weighting setting means 20a˜c, and outputs the added elasticity image data to the color scan converter 15.

Here, the weighting of the elasticity frame data will be described in concrete terms. The output signals of the weighted and added elasticity frame data is expressed, for example, by the formula below.

Out(i·j)=α·N(i·j)+β·(N-1)(i·j)+γ·(N-2)(i·j) [Formula 1]

Index i,j represents a coordinate on each set of elasticity frame data. The sum of α, β and γ is 1. The adder 21 performs addition processing of points on the same coordinate data based on the elasticity frame data “N”, elasticity frame data “N-1” and elasticity frame data “N-2” that are selected from the buffer memory. The elasticity frame data added by the above-mentioned addition processing is transmitted to the color scan converter 15 as elastic image data.

Next, a concrete example of the weighting in the first embodiment of the present invention will be described using FIG. 3.

In the present embodiment, setting of weighting or number of addition is performed using the pressure information measured by the pressure sensor 16 and the pressure measuring unit 17. FIG. 3 shows the embodiment, upon compressing the object 1, to change the weighting or number of addition of elasticity frame data, based on the pressure value measured in the pressure sensor or the pressure measuring unit 17.

As shown in FIG. 3 [symbol] a [symbol] , the pressure sensor 16 is attached to the end of the probe 2. When the object 1 is compressed using the probe 2 for obtaining an elastic image, the reflected echo signals are detected from the probe 2, and the electronic signals related to the pressure are transmitted by the pressure sensor 16 to the pressure measuring unit 17. The pressure measuring unit 17 calculates pressure information based on the electronic signals, and transmits the calculated pressure information to the weighting control unit 18. The weighting control unit 18 gives a command to the weighting setting means 20a˜c to perform the weighting in accordance with the pressure information.

The graph shown in FIG. 3 [symbol] b [symbol] displays the pressure values obtained by adding/reducing pressure to/from the object 1 by making them correspond to the time. By this graph, it is possible to recognize the compression condition of the object 1 in chronological order.

Time phase (0)˜time phase (3) where adequate compression is applied has small compression value, and is a phase wherein large displacement is generated by a small change in compression value between adjacent two frames. Time phase (4)˜time phase (7) where the compression turns back has a large compression value, and is a phase wherein small displacement is generated between adjacent two frames by a change in compression value. Intervals between the pressure measurement by the pressure sensor 16 should be the same as the frame rate where the RF signal frame data can be obtained, because the pressure value can be directly measured by corresponding to the respective time phases (0)˜(7).

For example, in the case that the measured pressure value is lower than “a” which is the case of time phases (0)˜(3), the weighting control unit 18 determines that the present frame 3 as a highly reliable elasticity frame data. In this manner, when the elasticity frame data 3 has high reliability, the weighting control unit 18 makes the value of multiplier coefficient (weight) α of the elasticity frame data 3 in the [formula 1] larger compared to the multiplier coefficient (weight) β or γ of other elasticity frame data, and outputs them respectively to the weighting setting means 20a˜c. For example, α is set as 0.8, β is set as 0.1 and γ is set as 0.1. Or, the pressure value may be obtained for each of elasticity frame data (3), elasticity frame data (2) and elasticity frame data (1), to obtain α, β and γ on the basis of the relative comparison of the respectively obtained values. In other words, the smaller the measured pressure value of the elasticity frame data is the value to be allotted to α, β and γ should be larger, and the larger the measured pressure value of the elasticity frame data is the value to be allotted to α, β and γ should be smaller.

Also, while the case exhibited above for evaluating reliability of the above-mentioned one or three sets of frames and changing the weighting of the three sets of elasticity frame data based on the evaluation thereof, there are cases that a specific elasticity frame data is evaluated as having high reliability and that it is not necessary to use other elasticity frame data for addition such as weighting. In such case, multiplier coefficient (weight) does not necessarily have to be allotted to all of α, β and γ of three elasticity frame data for addition, and the number of addition may be adjusted, for example, by setting γ as 0 and using only remaining two sets of elasticity frame data. Therefore, in the weighting control unit 20, not only weight but also the number of sets of elasticity frame data to be added can also be changed in accordance with the measurement result of the compression whereby making it possible to construct and display the most appropriate elastic image.

On the other hand, in the case that the measured pressure value is higher than “b” in time phases (4)˜(7), the weighting control unit 18 determines that the present frame 7 has low reliability. In this manner, when the elasticity frame data 3 has low reliability, the weighting control unit 20 makes the value of multiplier coefficient (weight) α of the elasticity frame data 7 (when N=7) in the [formula 1] smaller compared to the multiplier coefficient (weight) β or γ of other elasticity frame data, and outputs them respectively to the weighting setting means 20a˜c. For example, α is set as 0.2, β is set as 0.4 and γ is set as 0.4. Or, the pressure value may be obtained for the respective elasticity frame data (7), elasticity frame data (6) and elasticity frame data (5), to obtain α, β and γ on the basis of the relative comparison of the respectively obtained values.

In other words, for example, the larger the measured pressure value of the elasticity frame data is the value to be allotted to α, β and γ should be smaller, and the smaller the measured pressure value of the elasticity frame data is the value to be allotted to α, β and γ should be larger.

Also, while the case is exhibited above for evaluating reliability of the above-mentioned one or three sets of frames based on the pressure value upon acquiring the frame data thereof and changing the weighting of the three sets of elasticity frame data based on the evaluation of reliability, there are cases that a specific elasticity frame data is evaluated as having low reliability and that it is not necessary to use other elasticity frame data such as weighting for addition. In such case, multiplier coefficient (weight) does not necessarily have to be allotted to all of α, β and γ of three elasticity frame data, and the number of addition may be adjusted, for example, by setting α as 0 and using only remaining two sets of elasticity frame data. Therefore, not only weight but also the number of sets of elasticity frame data to be added can also be changed in accordance with the evaluation result of reliability in the weighting control unit 18 whereby making it possible to construct and display the most appropriate elastic image.

The weighting setting means 20a˜c performs weighting using the set α, β and γ, and performs addition of the plurality of elasticity frame data in the adder 21. Then the elastic image construction unit 14 outputs the added elasticity frame data as elastic image data.

Next, operation of the first embodiment in the present invention will be described referring to FIG. 4.

### (Step 22)

By the pressure measuring unit 17, the value of the pressure applied to the object from the probe upon acquiring the relative elasticity frame data is calculated.

### (Step 23)

Reliability of the elasticity frame data is determined based on how much smaller the pressure value acquired by the pressure measuring unit 17 is with respect to the threshold value.

### (Step 24)

When the condition of step 23 is satisfied, the weighting control unit 18 sets the weight of the present elasticity frame data to be high with respect to the weighting setting means 20a˜c. Or, after comparing the pressure values between the adjacent elasticity frame data, the weight of the elasticity frame data having a small pressure value is set to be high.

### (Step 25)

When the condition of step 23 is not satisfied, the weighting control unit 18 sets the weight of the present elasticity frame data to be low with respect to the weighting setting means 20a˜c. Or, after comparing the pressure values between the adjacent elasticity frame data, the weight of the elasticity frame data having a large pressure value is set to be low.

### (Step 26)

The adder 21 adds the plurality of elasticity frame data processed with weighting by the weighting setting means 20a˜c, and outputs the added elasticity frame data to the color scan converter 15.

### (Step 27)

The elastic image data converted in the color scan converter 15 is displayed on the image display device 10.

In accordance with the ultrasonic diagnostic apparatus related to the above-described first embodiment, the pressure value added to the object from the probe upon acquiring elasticity frame data is measured, and the weight of the elasticity frame data having low pressure value is made higher based on the result of the measurement. In other words, since the weight of the elasticity frame data having high reliability is made high, the elastic image data obtained by addition of the elasticity frame data is optimized more effectively. Also, since the number of sets of data for addition can be changed in accordance with the weighting rate in the present embodiment, it is possible to optimize the number of addition for improving reliability of the obtained elasticity frame data.

### Embodiment 2

Next, a second embodiment of the ultrasonic diagnostic apparatus related to the present invention will be described using the diagrams.

Here, the second embodiment will be described referring to FIG. 5. The second embodiment is different from the first embodiment in the point that weighting or adjustment of the number of data sets for addition is performed using positional information or moving information of the probe 2 caused by compression, using a sensor such as a magnetic sensor 28.

As shown in FIG. 5 [symbol] a [symbol] , the magnetic sensor 28 for detecting magnetic field is mounted in the probe 2 and detects high-frequency magnetic field irradiated from a magnetic field source 29. A position/direction analyzing unit which is not shown in the diagram is for obtaining the position or direction of the magnetic field sensor 28, that is probe 2 on the basis of the magnetic field source 29 by analyzing the magnetic detection signals detected by the magnetic field sensor 28 in the condition that the high-frequency magnetic field is irradiated by excitation of the magnetic field source 29. The position/direction analyzing unit is connected to the weighting control unit 18 and the image display device 10.

FIG. 5 [symbol] b [symbol] shows the position (moving distance) of the probe 2 detected by the magnetic sensor 28, and the range of movement of the probe 2. The detail thereof will be described below.

The range of movement “c” is set in advance as a compression range. The operator continually applies pressure using probe 2 so as to make the pressure value fall within this range of movement “c”. When the object 1 is compressed, the positional information of the probe 2 is displayed on the image display device 10. The positional information is the moving distance of the probe 2 in the depth direction (compressing direction) of the object 1.

As a concrete example, the case that the compression range is 10 mm will be described below. The range “d” from which is 2 mm to 8 mm is set or recorded as an adequate compression range, and the interval from 0 mm to 2 mm after starting the compression of the probe 2 or the range “e” of 8 mm˜10 mm which is before and after the compression turns back is set as a transition period. In the compression range “d”, since it is in the adequate compression zone, the weighting control unit 18 outputs each of the frame data to the weighting setting means 20a˜c respectively so that the multiplier coefficient (α, etc.) of the elasticity frame data “N” in [formula 1] turns out to be comparatively large. In the compression range “e”, since it is the zone where inadequate compression is applied, the weighting control unit 18 outputs each of the frame data to the weighting setting means 20a˜c respectively so that the multiplier coefficient (α) of the elasticity frame data “N” in [formula 1] turns out to be comparatively small. Or, with respect to each of the elasticity frame data for adding, the weight value of α, β and γ may be set by evaluating whether the position of the probe is in transition period or adequate range and by relatively comparing the evaluation result thereof using the plurality of elasticity frame data. The weighting setting means 20a˜c performs weighting by the set α, β and γ, and performs addition of the plurality of elasticity frame data in the adder 21. Then the elastic image construction unit 14 outputs the added elasticity frame data as elastic image data.

Next, operation of a second embodiment will be described referring to the diagrams. Except replacing the step 23 shown in FIG. 4 with determining reliability of elastic frame data based on whether the position of the probe 2 falls within a predetermined range or not, the operation is the same as the first embodiment. Thus the description on the areas of overlap will be omitted.

The ultrasonic diagnostic apparatus related to the above-described second embodiment has an advantage that the elastic image data obtained by addition of elasticity frame data can be optimized effectively and improved, since the position of the probe upon acquisition of each elastic frame data is evaluated, and based on the evaluation thereof, the weighting is made higher when the position of the probe is in a predetermined range and the weighting is made lower when the position of the probe is not in the predetermined range. Further in the present embodiment, for example, by setting the weighting of one set of elasticity frame data as 0 to make the number of data sets for addition variable, the number of data sets to perform addition for improving reliability of the acquired elastic image data can also be optimized.

### Embodiment 3

Here, a third embodiment will be described referring to FIG. 6. The difference from the first˜second embodiments is to optimize the weighting of elasticity frame data to add and the number of sets of data for the addition, by the frame rate for obtaining RF signals. In the present embodiment, two sets of frame data are weighted and added in accordance with the frame rate, or more than three sets of elasticity frame data are to be weighted and added.

For example, when the frame rate is high, since the displacement to be measured in the displacement measuring unit 12 is minimal, there are cases that it is difficult to measure the strain in the elasticity information calculating unit 13. This could lead to causing many errors or artifacts in the plurality of elasticity frame data. Therefore, there is a need for adding many sets of elasticity frame data to output as elastic image data, in order to reduce the influence of errors or artifacts.

Given this factor, in the present embodiment, a switch 31 is provided for selecting continued five sets of elasticity frame data in chronological order and recording the selected frame data to the buffer memory 30a˜buffer memory 30e. The switch 31 is connected to the weighting control unit 18, and performs control over the weighting setting means 20a˜weighting setting means 20e by the command from the weighting control unit 18.

In concrete terms, in the case of constructing stable elastic images by applying high frame rate with respect to the tissues having precipitous motion due to a factor such as beating, the switch 31 is controlled so that more than three, for example, five sets of elasticity frame data continued in chronological order are recorded to the buffer memory 30a˜buffer memory 30e. Also, in the case that compression can be made from the probe 2 and stable elastic images can be easily constructed to some extent with respect to the frame rate, the switch 31 is to be controlled so that two sets of elasticity frame data continued in chronological order are recorded to the buffer memory 30a˜buffer memory 30b.

The weighting setting means 20a˜20e performs a predetermined weighting on a plurality of elasticity frame data selected by the switch 31 and recorded in the respective buffer memories, and performs addition of the plurality of elasticity frame data in the adder 21. Then the elastic image construction unit 13 outputs the added elasticity frame data as elastic image data.

In this embodiment, elastic image data can be selected also by adjusting the weighting of more than 3 sets (5 sets here) of elasticity frame data and including the data having the weight which is zero, without using the switch 31. The above-mentioned selection of the number of sets of elasticity frame data and weighting, etc. will be described concretely. The output signals of the elasticity frame data can be expressed by the formula below.

Out(i,j)=α·N(i,j)+β·(N-1)(i,j)+γ·(N-2)(i,j)+δ·(N-3)(i,j)+ε·(N-4)(i,j) [Formula 2]

The index “i,j” represents the coordinate of the respective frame data. The sum of α, β, γ, δ and ε is 1.

In the case that the frame rate is high, multiplier coefficients α, β, γ, δ and ε of the elasticity frame data “M” in the formula 2 are respectively equalized and outputted to the weighting setting means 20a˜e. For example, it is set as α=β=γ=δ=ε=0.2. Then the weighting setting means 20a˜e perform weighting by the set multiplier coefficients, and perform addition of the plurality of elasticity frame data in the adder 21. The elastic image construction unit 13 outputs the added elasticity frame data as elastic image data.

In the case that the frame rate is low, a value is provided to multiplier coefficients α and β of the elasticity frame data “M” in the formula 2, and the multiplier coefficients γ, δ and ε are made 0, and outputted respectively to the weighting setting means 20a˜e. For example, it is set as α=β=0.5, γ=δ=ε=0. Then the weighting setting means 20a˜e perform weighting by the set multiplier coefficients, and perform the addition of the plurality of elasticity frame data in the adder 21. The elastic image construction unit 13 outputs the added elasticity frame data as elastic image data.

Also, in the case that the frame rate is high, the weighting may be performed so as to make the multiplier coefficients β and δ of the weighting setting means 20b and the weighting setting means 20d become 0, so that the elasticity frame data is alternately recorded to the buffer memory 30a, buffer memory 30c and the buffer memory 30e.

While the pattern for performing weighting using five buffer memories is described in the third embodiment, buffer memories and the weighting means corresponding thereto may be more than five, and may be arbitrarily modified.

Next, the operation of the third embodiment will be described referring to the diagrams. The operation of the third embodiment is the same as the operation of the first embodiment except that the step 22 shown in FIG. 4 is replaced with the step for determining reliability of elastic frame data based on whether the frame rate for acquiring RF signals is fast or slow. Thus the areas of overlap will be omitted.

The ultrasonic diagnostic apparatus related to the above-described third embodiment has an advantage that the reliability of elastic images to be displayed is improved since the number of sets of elasticity frame data obtained by addition of elasticity frame data can be better optimized by evaluating the frame rate upon acquisition of elasticity frame data and, based on the evaluation result, adjusting display images through increasing the number of data sets for addition in the case that the frame rate is high and decreasing the number for addition in the case that the frame rate is low. Also, in this embodiment, the weighting upon addition of the respective elasticity frame data may be changed by the frame rate obtained when the elasticity frame data is acquired, which makes it possible to improve the reliability of elasticity frame data to be obtained from the scan plane thereof.

### Embodiment 4

Next, a fourth embodiment of the present invention will be described referring to FIG. 7.

FIG. 7 [symbol] a [symbol] shows the pattern wherein an elastic image is displayed on the image display device 10 by repeating addition/reduction of pressure to/from the object 1. In the graph of FIG. 7 [symbol] b [symbol] , the displacement being obtained in the displacement measuring unit 12 by adding/reducing pressure to/from the object 1 is displayed by making it correspond to time. By this graph, compression condition of the object 1 can be recognized in chronological order.

When pressure is added to object 1 by the probe 2, the shape of the object 1 is changed and bent up to a displacement limit value “f”. Then by pulling the probe 2 at the displacement limit value “f” and reducing pressure from the object 1, the object 1 returns to the original shape. Here, the cycle that the object 1 is returned from a certain shape to the original shape (for example, time phase 0˜time phase α) is set as compression cycle.

In time phase (0)˜time phase (3) of the above-described pressure cycle is the range where pressure can be applied freely since the value is apart from the displacement limit value “f”. It is the zone where pressure is to be applied to the object 1 in full measure. Therefore, the average value of the displacement calculated by the time phase (0) and the time phase (1), the average value of the displacement calculated by the time phase (1) and the time phase (2) and the average value of the displacement calculated by the time phase (2) and the time phase (3) are substantially large values. Thus the average value of change in strain to be calculated among the time phase (0)˜time phase (1), time phase (1)˜time phase (2) and time phase (2)˜time phase (3) comes out as a comparatively large value.

For example, the elasticity frame data acquired based on the RF signal frame data of the time phase (2)˜time phase (3) selected in the RF signal frame data selecting unit 11 is set as elasticity frame data (3). Also, the elasticity frame data acquired based on the RF signal frame data of the time phase (1)˜(2) selected in the RF signal frame data selecting unit 11 is set as elasticity frame data (2). Also, the elasticity frame data acquired based on the RF signal frame data of the time phase (0)˜time phase (1) selected in the RF signal frame data selecting unit 11 is set as elasticity frame data (1).

Also, in the frame data (3˜1), for example, the average value of the displacement in a predetermined region of interest is set as DA(3)˜DA(1), and the average value of the change in strain is set as SA(3)˜SA(1).

Here, the weighting control unit 18 sets a threshold value for determining the quality of elasticity frame data, and if the average value of the displacement calculated in the displacement measuring unit 12 or the average value of the strain calculated in the elasticity information calculating unit 13 is larger than the set threshold value (for example, about 0.5%), the elasticity frame data (3˜1) is determined as having high quality. Because if the strain is more than 0.5, it can be considered that approximately linear relationship is maintained between the stress and the strain.

In concrete terms, the weighting control unit 18 sets “K” as the threshold value with respect to the average value of the displacement in the relative elasticity frame data (for example, frame 3), “K′” as the threshold value of the difference related to how large the average value of the displacement in the relative elasticity frame data is to the average value of the displacement of the previous frame, “L” as the threshold value with respect to the average value of the change of strain in the relative elasticity frame data, and “L′” as the threshold of the difference related to how large the average value of the change of strain in the relative elasticity frame data is to the average value of the change of strain in the previous frame. Then it determines whether the relative elasticity frame data is a high quality image or not based on the following discriminants [formula 3]˜[formula 6].

The following formulas are set as:

DA(3)>K [Formula 3]

DA(3)−DA(2)>K′ [Formula 4]

SA(3)>L [Formula 5]

SA(3)−SA(2)>L′ [Formula 6]

When all or some of the above [formula 3]˜[formula 6] are satisfied, the weighting control unit 18 determines that the present frame 3 is the elasticity frame data having a high quality. In this manner, when reliability of the elasticity frame data 3 is high, the weighting control unit 18 makes the value of multiplier coefficient (weight) α of the elasticity frame data 3 in the [formula 1] larger compared to the multiplier coefficient (weight) β or γ of the other elasticity frame data and outputs them to the weighting setting means 20a˜c respectively. For example, α is set as 0.8, β is set as 0.1 and γ is set as 0.1.

Or, with respect to each of the elasticity frame data (3), the elasticity frame data (2) and the elasticity frame data (1), the difference related to how large the average value of the displacement of the relevant elasticity frame is to the average value of the displacement of the previous frame and the difference related to how large the average value of the change in strain of the relevant elasticity frame data is to the average value of the change in strain of the previous frame may be acquired so as to obtain α, β and γ based on the relevant comparison of the acquired values.

In other words, for example, the larger value of α, β and γ should be allotted to the elasticity frame data having the larger value in the left part obtained by [formula 3]˜[formula 6], and the smaller value of α, β and γ should be allotted to the elasticity frame data having the smaller value in the left part of the formulas.

On the other hand, in this compression cycle, the time phase (4)˜time phase (7) is close to the displacement limit value “f”, thus it is the range where the pressure can not be freely applied, that is the zone where the pressure can not be added substantially to the object 1. Therefore, the average value of the displacement calculated by the time phase (6) and the time phase (7), the average value of the change in displacement calculated by the time phase (5) and the time phase (6) and the average value of the displacement calculated by the time phase (4) and the time phase (5) are not very large. In this case, the average value of the change in strain among the time phase (6)˜time phase (7), the time phase (5)˜time phase (6) and the time phase (4)˜time phase (5) is calculated as a comparatively small value.

For example, the elasticity frame data obtained based on the RF signal frame data of the time phase (6)˜time phase (7) selected in the RF signal frame data selecting unit 11 is set as the elasticity frame data (7). Also, the elasticity frame data acquired based on the RF signal frame data of the time phase (5)˜time phase (6) selected in the RF signal frame data selecting unit 11 is set as the elasticity frame data (6). Also, the elasticity frame data acquired based on the RF signal frame data of the time phase (4)˜time phase (5) is set as the elasticity frame data (5).

Also in the elasticity frame data (7˜5), for example, the average value of the displacement in a predetermined region of interest is set as DA(7)˜DA(5), and the average value of the change in strain is set as SA(7)˜(5).

Here, the weighting control unit 18 sets a threshold value for determining quality of the elasticity frame data, and determines that the elasticity frame data (3˜1) does not have high quality if the average value of the displacement calculated in the displacement measuring unit 12 or the average value of the strain calculated in the elasticity information calculating unit 13 is smaller than the set threshold value (for example, about 0.5%). Because it can be considered that the relationship which is approximately linear can not be maintained between the stress and the strain when the strain is 0.5% or smaller. For example, there is a possibility that the pressure to the object 1 expressed in the time phase thereof is not added evenly in the vertical direction to the pressure compression surface of the probe, but added unequally to the oblique direction to the compression surface of the probe. In such a case that the elasticity frame data calculated when the pressure was applied unequally to the object 1 is outputted as it is to the color scan converter 15, noncontiguous portions will be generated in the time change of the stress distribution in a series of elasticity frame data in time axis direction. In this case, since the pressure can not be applied adequately to the object 1 in the time phase (4)˜time phase (7), there are many occasions that the elasticity frame data that is useful as diagnostic images can not be generated.

In concrete terms, the weighting control unit 18 sets a threshold value “K” as the threshold value with respect to the average value of the displacement of the relevant elasticity frame data (for example, a frame 3), a “K′” as the threshold value of the difference regarding how small the average value of the displacement of the relative elasticity frame data is with respect to the average value of the displacement of the previous frame, an “L” as the threshold value with respect to the average value of the change in strain of the relevant elasticity frame data, and an “L′” as the threshold of the difference regarding how small the average value of the strain in change of the relevant elasticity frame data is with respect to the average value of the change in strain of the previous frame. Then the determination is to be made whether the relevant elasticity frame data is reliable or not based on the following discriminants [formula 7]˜[formula 10].

DA(7)<K [Formula 7]

DA(7)−DA(6)<K′ [Formula 8]

SA(7)<L [Formula 9]

SA(7)−SA(6)<L′ [Formula 10]

When all or some of the above [formula 7]˜[formula 10] are satisfied, the weighting control unit 218 determines that the present frame 7 is the elasticity frame data having low quality. In this manner, when the elasticity frame data 3 has low reliability, the weighting control unit 18 makes the value of multiplier coefficient (weight) α of the elasticity frame data 3 in the [formula 1] smaller compared to β or γ, and outputs them to the weighting setting means 19a˜c respectively. For example, α is set as 0.2, β is set as 0.4 and γ is set as 0.4. Or, with respect to each of the elasticity frame data (7), the elasticity frame data (6) and the elasticity frame data (5), the difference regarding how large the average value of the displacement of the relevant elasticity frame is to the average value of the displacement of the previous frame and the difference regarding how large the average value of the change in strain of the relevant elasticity frame data is to the average value of the change in strain of the previous frame may be acquired so as to obtain α, β and γ based on the relevant comparison of the acquired values.

In other words, for example, the smaller the value of the left part obtained in [formula 3]˜[formula 6] is the value to be allotted to α, β and γ should be smaller, and the larger the value of the left part is the value to be allotted to α, β and γ should be smaller.

Also, while the case for evaluating reliability of the above-mentioned one or three sets of frames and changing the weighting of the three sets of elasticity frame data based on the evaluation thereof, there are cases that a specific elasticity frame data is determined as having low reliability whereby it is unnecessary to use other elasticity frame data for addition such as weighting. In such case, the weight such as 0.1 does not necessarily have to be allotted to all of α, β and γ of three elasticity frame data, and the number of data sets for addition may be adjusted, for example, by setting α as 0 and using only the remaining two sets of elasticity frame data. Therefore, optimum elastic images can be constructed and displayed by changing not only the weighting but also the number of sets of elasticity frame data to be added in accordance with the evaluation result of reliability performed in the weighting control unit 18.

Next, operation of a fourth embodiment will be described referring to the diagrams. Except for replacing the (step 22) in FIG. 4 with determining reliability of the elasticity frame data based on the above-described [formula 2]˜[formula 10], the operation is the same as the first embodiment. Thus the description on the areas of overlap will be omitted.

The above-description of specific embodiments is not intended to limit the present invention to the particular forms described, but on the contrary, the intension is to cover all modifications, equivalents, and alternations falling within the spirit and scope of invention. For example, while the pressure to be applied from the probe to the object is measured using the pressure sensor 16 and the pressure measuring unit 17 for evaluating reliability of the elasticity frame data for adding in the above-mentioned embodiment 1, the method for measuring the pressure does not have to be limited thereto. For example, the method disclosed in JP-A-2005-66041 may be used. More concretely, the pressure may be measured by placing a deformable body for pressure measurement between the object and the probe and obtaining the deformation quantity or the change in thickness of the deformable body. Also, it is needless to say that the method described in the first embodiment˜fourth embodiment can be used independently or by combining two or more embodiments. For example, from information on pressure to be applied from the probe to the object, information on moving distance or position of the probe, frame rate, elasticity frame data, etc., the information acquired by calculation may be used independently for reliability evaluation of elasticity frame data, or by combining a plurality of information to secure the reliability of the evaluation result. Also, the number of elasticity frame data to be the target for reliability evaluation can be any number as long as it is more than one. Or, the threshold value described above for evaluating reliability may be stored in advance by an operator in a device such as memory of the ultrasonic diagnostic apparatus. Also, the threshold value to be used for evaluating reliability in the above-described embodiments may be stored in advance through the input and setting by an operator, in a device such as a memory of the ultrasonic diagnostic apparatus.

Also, it may be set so that the above-described evaluation result of reliability can be displayed on the image display unit 10. Also, while one threshold value may be set on information such as a pressure value measured for the above-described reliability evaluation so as to determine the above-described weighting value by comparing the pressure value to the set threshold value, the number of threshold value may be two or more and they may be converted into a table-like chart indicating the list of weighting values corresponding to information such as pressure values.

1. An ultrasonic diagnostic apparatus comprising:
an ultrasonic probe;
frame data acquiring means for compressing target tissues of an object to be examined by the ultrasonic probe, and acquiring a plurality of RF signal frame data in chronological order during the process that the compression condition of the target tissues is being changed;
elasticity information calculating means for taking out a pair of frame data from among the plurality of RS signal frame data, and generating a plurality of elasticity frame data by performing calculation on the strain or elasticity modulus of the respective positions in the target tissues;
elastic image constructing means for constructing elastic images by adding the generated plurality of elasticity frame data; and
display means for displaying the constructed elastic images,
characterized in further comprising:
evaluation means for evaluating reliability of the plurality of elasticity frame data to be added based on the degree of the compression.

2. The ultrasonic diagnostic apparatus according to claim 1, characterized in comprising adjusting means for adjusting the addition of the plurality of elasticity frame data in accordance with the evaluation result by the evaluation means.

3. The ultrasonic diagnostic apparatus according to claim 2, wherein the adjusting means adjusts the addition by changing the weighting of the elasticity frame data to be the target for addition.

4. The ultrasonic diagnostic apparatus according to claim 2, wherein the adjusting means adjusts addition by adjusting the number of elasticity frame data for adding.

5. The ultrasonic diagnostic apparatus according to claim 1, characterized in comprising pressure measuring means for measuring the pressure between the ultrasonic probe and the object, wherein the evaluation means evaluates the reliability based on the measurement result of the pressure value by the pressure measuring means.

6. The superconducting diagnostic apparatus according to claim 5, wherein the pressure measuring means is a pressure sensor placed between the ultrasonic probe and the object.

7. The ultrasonic diagnostic apparatus according to claim 5, wherein the pressure measuring means is a deformable body for pressure measurement placed between the ultrasonic probe and the object, and calculates the pressure value by measuring the displacement amount of the deformable body for pressure measurement.

8. The ultrasonic diagnostic apparatus according to claim 5, wherein the evaluation means determines that the reliability is low when the pressure value is higher than an arbitrary threshold value, and determines that the reliability is high when the pressure value is lower than the arbitrary threshold value.

9. The ultrasonic diagnostic apparatus according to claim 2, wherein the evaluation means perform evaluation on all of the plurality of elasticity frame data to be the targets for addition.

10. The ultrasonic diagnostic apparatus according to claim 2, wherein the adjusting means, on the basis of the evaluation result on all of the plurality of elasticity frame data to be the targets for addition, performs addition by making the weighting high on the elasticity frame data having high reliability and making the weighting low on the elasticity frame data having low reliability.

11. The ultrasonic diagnostic apparatus according to claim 10, wherein the adjusting means adjusts the number of the plurality of elasticity frame data to be the targets for addition by including the weighting of which the value is zero in the weighting with respect to the plurality of elasticity frame data.

12. The ultrasonic diagnostic apparatus according to claim 2, wherein the adjusting means comprises frame data selecting means for adjusting the number of elasticity frame data by selecting the plurality of elasticity frame data to be the targets for addition.

13. The ultrasonic diagnostic apparatus according to claim 2, wherein the frame data selecting means adjusts the number of frame data for addition by thinning out the plurality of elasticity frame data aligned in chronological order.

14. The ultrasonic diagnostic apparatus according to claim 1, wherein the number of the plurality of elasticity frame data to be the targets for evaluation by the evaluation means is more than three.

15. The ultrasonic diagnostic apparatus according to claim 1, wherein the evaluation means comprises an ultrasonic probe moving distance measuring means for measuring the spatial moving distance of the ultrasonic probe upon compressing the target tissues or the position of the ultrasonic probe, and evaluates the reliability by adding the information regarding the measured moving distance or the position of the probe.

16. The ultrasonic diagnostic apparatus according to claim 15, wherein the ultrasonic probe moving distance measuring means is provided with either a magnetic sensor or a magnetic source on the ultrasonic probe, comprises the other one of the magnetic sensor or the magnetic source at the position other than the ultrasonic probe, and the magnetic sensor calculates the moving distance or the position of the probe by detecting magnetic intensity and/or direction of the magnetic field emitted from the magnetic source.

17. The ultrasonic diagnostic apparatus according to claim 15, wherein the evaluation means has storage means for storing a threshold value in relation to the moving distance or the position of the probe, and evaluates the reliability depending on the range determined by the threshold value to which the moving distance or the position of the probe belong.

18. The ultrasonic diagnostic apparatus according to claim 1, wherein the evaluation means comprises frame rate evaluating means for evaluating the reliability based on the frame rate upon acquisition of the RF signal frame data for generating the plurality of elasticity frame data, and evaluates the reliability by adding the information related to the frame rate.

19. The ultrasonic diagnostic apparatus according to claim 1, wherein the evaluation means comprises calculation means for performing calculation process with respect to the plurality of elasticity frame data, and evaluates the reliability of elasticity frame data by adding the information on how large or small the calculation result by the calculation means is with respect to an arbitrary threshold value.

20. The ultrasonic diagnostic apparatus according to claim 1, characterized in that the evaluation result by the evaluation means is displayed in conjunction with the display means.
